(19) United States
(12) Patent Application Publication
(10) Pub. No.: US 2026/0345514 A1
(43) Pub. Date: Nov. 5, 2026

(54) STABLE ROTIGOTINE TRANSDERMAL ADMINISTRATION KIT, AND PREPARATION METHOD THEREFOR AND USE THEREOF

(71) Applicant: NOVASTAGE PHARMACEUTICALS SHENZHEN, LTD, Shenzhen, Guangdong (CN)

(72) Inventor: Jiansheng TANG, Shenzhen, Guangdong (CN)

(21) Appl. No.: 19/103,759

(22) PCT Filed: Jan. 4, 2023
(86) PCT No.: PCT/CN2023/070543

§ 371 Date: Feb. 13, 2025

(51) Int. Cl. A61K 9/70; A61J 3/00; A61K 31/381; A61M 5/142

(52) U.S. Cl. CPC A61K 9/7061; A61J 3/00; A61K 9/7053; A61K 31/381; A61M 5/14248; A61M 2207/00

(57) ABSTRACT

Provided is a stable rotigotine transdermal drug delivery kit, comprising a transdermal drug delivery patch, wherein the transdermal drug delivery patch comprises: 1) a backing layer; 2) a matrix layer, comprising rotigotine or a pharmaceutically acceptable salt thereof that is dispersed in the matrix layer in a non-crystalline state, a stabilizer for inhibiting drug crystallization, an antioxidant and a pressure-sensitive adhesive; and 3) a release layer. Further provided is the preparation method for and use of the kit. The transdermal drug delivery kit maintains the content of related substances such as oxidized impurities in the transdermal patch at an extremely low level.

### TECHNICAL FIELD

The present invention relates to a stable rotigotine transdermal drug delivery kit. More specifically, the present invention relates to a transdermal drug delivery kit for rotigotine or a pharmaceutically acceptable salt thereof, and a preparation method therefor and use thereof.

### BACKGROUND ART

The transdermal drug delivery route is superior to the oral drug delivery route, and maintains drug concentration in the blood at a constant level by continuously delivering drugs to the systemic blood system. The transdermal drug delivery route not only reduces the peak-to-trough fluctuations in drug concentration in the blood, but also avoids the first-pass effect. In addition, since the transdermal drug delivery route avoids direct contact of drugs and excipients with the gastrointestinal system, side effects such as nausea and vomiting that are often associated with the oral drug delivery route are significantly reduced or eliminated. Another advantage of the transdermal drug delivery route lies in being not affected by diet. Drug delivery can be easily terminated when necessary by removing the transdermal patch from the skin. Furthermore, transdermal patches improve patient compliance by reducing the frequency of drug delivery. This is especially important for elderly patients and pediatric patients.

In recent years, the advantages of transdermal drug delivery have enabled many drugs to be effectively delivered via a transdermal route. These advances include the development of many physical methods for increasing skin permeability and facilitating transdermal drug delivery, e.g., using iontophoresis, electroporation, ultrasound, or microneedle. However, drugs that can be effectively and safely applied through the skin for 7 days or more without causing skin adhesion, skin irritation or sensitization are still limited.

Rotigotine is a compound (−)-5,6,7,8-tetrahydro-6-[propyl-[2-(2-thienyl)ethyl]-amino]-1-naphthol (INN) having a structure as shown below:

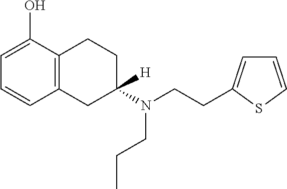 [SMILES: CCCN(CCc1cccs1)[C@H]1CCc2c(O)cccc2C1]

Rotigotine is disclosed as an active agent for treating patients with Parkinson's disease, Parkinson's plus syndrome, depression and restless legs syndrome, as well as an active agent for treating or preventing dopaminergic neuron loss and for treating pain.

Known pharmaceutical compositions containing rotigotine include transdermal therapeutic systems or patches (TTS), depot forms, iontophoresis devices and intranasal formulations.

To date, different transdermal therapeutic systems (TTS) for the administration of rotigotine have been described.

WO 94/07468 discloses a transdermal therapeutic system (TTS) containing rotigotine hydrochloride as an active substance in a two-phase matrix, wherein the two-phase matrix is essentially formed of a hydrophobic polymer material as a continuous phase and a dispersed hydrophilic phase contained therein, and the hydrophilic phase mainly contains the drug and hydrated silicon dioxide. It is believed that silicon dioxide may increase the maximum possible loading with the hydrophilic salt in TTS. The formulations of WO 94/07468 generally contain additional hydrophobic solvents, penetration-enhancing substances, dispersing agents and especially emulsifiers. The emulsifiers are used to emulsify an aqueous solution of an active component in a lipophilic polymer phase. TTS prepared using such system has been tested in healthy subjects and Parkinson's disease patients. However, satisfactory drug plasma levels have not been achieved.

WO 99/49852 describes a different transdermal therapeutic system, comprising: a backing layer which is inert to matrix components, a self-adhesive matrix layer containing an effective amount of rotigotine or rotigotine hydrochloride, and a protective film to be removed prior to use. The matrix system consists of an acrylate-based or silicone-based non-aqueous polymer adhesive system.

U.S. Pat. No. 9,925,150B2 discloses a solid dispersion consisting of a silicone pressure-sensitive adhesive as a dispersing agent and soluble polyvinylpyrrolidone and rotigotine as a dispersed phase, wherein the weight ratio of rotigotine to polyvinylpyrrolidone ranges from about 9:3.5 to about 9:6. It has been found in comparative examples 1-3 of the present application that such solid dispersions are unstable because rotigotine often crystallized out from the solid dispersions. Furthermore, the skin adhesion of the silicone pressure-sensitive adhesive matrix is not as good as expected.

U.S. Pat. No. 9,226,902B discloses a transdermal drug delivery system for amorphous rotigotine, wherein the stabilizer is soluble polyvinylpyrrolidone, a polyvinylpyrrolidone-vinyl acetate copolymer, hydroxypropyl cellulose, hydroxypropyl methyl cellulose, hydroxyethyl cellulose, ethyl cellulose, or a combination thereof, and the weight ratio of the stabilizer to rotigotine is about 0.5 or more.

In general, it is well known in the art that stabilizing an amorphous state of a medicinal substance in a pharmaceutical dosage form (including a transdermal system) is very difficult. The amorphous form of rotigotine is only relatively stable and is easily converted into a crystalline form in a transdermal formulation.

It has been found that the above-mentioned systems unfortunately exhibit problems regarding long term stability. If rotigotine crystals form in the self-adhesive matrix during long-term storage, the growth of the crystals may lead to a reduced release rate of rotigotine, with the risk of eventually falling below a specified value.

Due to the appearance of a novel polymorphic form (form II) of rotigotine, crystals may form on commercial patches. Attempts to modify production processes have achieved only very limited success. These patches still require low temperature storage before application to the skin. This results in more complex application instructions being required for rotigotine patches. For example, the cooled pharmaceutical product should be taken out of the refrigerator at least 1 hour before application, and the adhesive on the patch must be heated when the patch is applied.

None of the prior art discloses a method for improving the stability of a rotigotine transdermal drug delivery formulation. Therefore, there is an urgent need for a rotigotine transdermal drug delivery system that can keep active substances stable for a long period of time, maintain the content of related substances in an extremely low range, and meet the requirements of ICH Q3B.

### SUMMARY OF THE INVENTION

It has been found in the present application that there is a problem regarding crystallization of rotigotine in a transdermal patch formulation. In order to inhibit the crystallization of rotigotine, the crystallization inhibitors povidone, copovidone and crospovidone CL-M are used in the patch formulation in the present application. Soluble povidone alone is not sufficient to inhibit the crystallization of rotigotine in the transdermal patch formulation. The problem regarding crystallization of rotigotine in a transdermal patch can be effectively suppressed by replacing part or all of the water-soluble povidone with crospovidone.

It has been further found in the present application that rotigotine is particularly unstable in a transdermal patch formulation containing a peroxide excipient. The peroxide excipient includes, for example, an acrylic pressure-sensitive adhesive, povidone, copovidone, and crospovidone CL-M. Under conventional storage conditions for pharmaceutical products, rotigotine in a transdermal patch formulation is easily degraded by peroxides to produce N-oxides (EP impurity E), which are further decomposed into other related substances (such as EP impurity C, EP impurity B, and EP impurity K), rendering the transdermal patch formulation unusable.

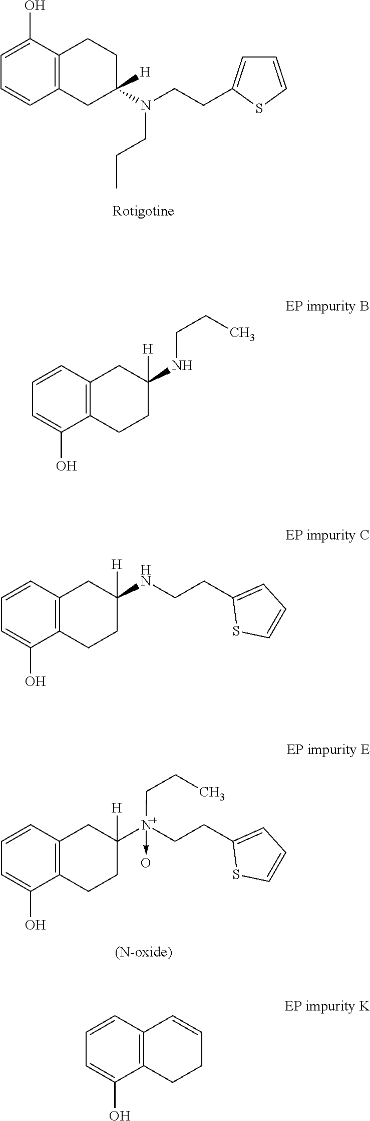

The present inventors have also found that adding an antioxidant combination to a drug matrix layer of a transdermal patch can reduce the generation of related substances and inhibit the degradation of rotigotine by peroxides.

The present inventors have also unexpectedly found that filling a packaging container (e.g., a packaging bag) of a patch formulation, to which an antioxidant is added as a matrix, with an inert gas such as nitrogen can further inhibit the degradation of rotigotine.

The present applicants have surprisingly found that, on the basis of filling with nitrogen, adding a hygroscopic agent not in contact with the matrix layer of the patch to a packaging can further inhibit the oxidative degradation of rotigotine.

The present inventors have also unexpectedly found that on the basis of filling with nitrogen or a combination of filling with a gas and a hygroscopic agent in contact with the matrix layer of the patch, further addition of an oxygen scavenger not in contact with the matrix layer of the patch to the packaging bag does not result in decreased stability. Unexpectedly, a hygroscopic agent not in contact with the matrix layer of the patch reduces the generation of oxidative impurities.

Therefore, an object of the present invention is to provide a stable matrix-type rotigotine transdermal drug delivery formulation and a kit thereof, in which the rotigotine does not crystallize after being stored for a period of time.

An object of the present invention is to provide a stable matrix-type rotigotine transdermal drug delivery formulation and a kit thereof, which, after storage for a period of time, maintain the content of related substances at an extremely low level and can continuously deliver rotigotine or a pharmaceutically acceptable salt thereof at a therapeutically effective blood drug level over a prolonged period of time.

An object of the present invention is to provide a stable matrix-type rotigotine transdermal drug delivery formulation and a kit thereof, which can inhibit the degradation of rotigotine by peroxides in excipients.

Another object of the present invention is to provide a method for preparing a matrix-type rotigotine transdermal drug delivery formulation and a kit thereof. After storage for a period of time, the matrix-type rotigotine transdermal drug delivery formulation and kit thereof prepared by the method have no crystallization of rotigotine, maintain the content of related substances at an extremely low level, and can continuously deliver rotigotine or a pharmaceutically acceptable salt thereof at a therapeutically effective blood drug level over a prolonged period of time.

Another object of the present invention is to provide a method for treating or preventing a disease sensitive to the action of a dopamine receptor agonist, comprising administering a therapeutically effective amount of a matrix-type crystallization-inhibiting rotigotine transdermal drug delivery kit to a subject in need thereof.

Another object of the present invention is to provide a method for treating or preventing a disease sensitive to the action of rotigotine, comprising administering a therapeutically effective amount of a matrix-type crystallization-inhibiting rotigotine transdermal drug delivery kit to a subject in need thereof.

Another object of the present invention is to provide a method for treating or preventing Parkinson's disease, Parkinson's plus syndrome, depression, restless legs syndrome, pain and dopaminergic neuron loss, comprising administering a therapeutically effective amount of a matrix-type crystallization-inhibiting rotigotine transdermal drug delivery kit to a subject in need thereof.

Another object of the present invention is to provide the use of a therapeutically effective amount of a matrix-type crystallization-inhibiting rotigotine transdermal drug delivery kit in the preparation of a medicament for treating or preventing a disease sensitive to the action of a dopamine receptor agonist.

Another object of the present invention is to provide the use of a therapeutically effective amount of a matrix-type crystallization-inhibiting rotigotine transdermal drug delivery kit in the preparation of a medicament for treating or preventing a disease sensitive to the action of rotigotine.

Another object of the present invention is to provide the use of a therapeutically effective amount of a matrix-type crystallization-inhibiting rotigotine transdermal drug delivery kit in the preparation of a medicament for treating or preventing Parkinson's disease, Parkinson's plus syndrome, depression, restless legs syndrome, pain and dopaminergic neuron loss.

### DETAILED DESCRIPTION OF THE INVENTION

A first aspect of the present invention provides a rotigotine transdermal drug delivery kit, comprising a transdermal drug delivery patch, wherein the transdermal drug delivery patch comprises:
1) a backing layer;
2) a matrix layer, comprising rotigotine or a pharmaceutically acceptable salt thereof that is dispersed in the matrix layer in a non-crystalline state, a stabilizer for inhibiting drug crystallization, an antioxidant and a pressure-sensitive adhesive; and
3) a release layer;
wherein the transdermal drug delivery patch is packaged in a packaging container.

In some embodiments, the packaging container is filled with an inert gas. The inert gas is preferably nitrogen. Preferably, nitrogen is filled to reduce oxygen to below about 3.0%, and more preferably, the inert gas is filled to reduce oxygen to below about 2.0%, 1.0%, 0.5%, 0.2%, 0.15%, 0.1%, 0.05% and 0.01%.

In some embodiments, the stabilizer for inhibiting drug crystallization contains a peroxide; preferably, the peroxide-containing stabilizer for inhibiting drug crystallization comprises insoluble crospovidone; further preferably, the insoluble crospovidone is selected from one or more of insoluble crospovidone CL-M, crospovidone CL, crospovidone CL-F and crospovidone CL-SF.

In some embodiments, in addition to the insoluble crospovidone, the stabilizer for inhibiting drug crystallization further comprises soluble povidone, wherein the soluble povidone is preferably one or more of povidone K30, povidone K90, povidone K12, povidone K17, povidone K25, plasdone K29/32, and copovidone VA64.

In some embodiments, the weight ratio of rotigotine to insoluble povidone is no more than 9:40, preferably 9:1-9:24, 9:5-9:22.8, 9:5, 9:6.8, 9:8, 9:10, 9:12, 16.5, 9:18, 9:20, 9:21, 9:22.8, 9:24 and 9:27.

In some embodiments, the weight ratio of rotigotine to soluble povidone is 9:0.5-9:10, and specifically, can be selected from 9:1, 9:2, 9:3, 9:4, 9:5, 9:6, 9:7, 9:8, 9:9 and 9:10.

In some embodiments, a hygroscopic agent not in contact with the matrix layer of the patch is further added to the packaging container. The hygroscopic agent is selected from one or more of calcium chloride, calcium oxide, barium oxide, calcium sulfate, molecular sieve, sodium sulfate, magnesium sulfate, silicon dioxide, silica gel, potassium hydroxide, potassium carbonate and phosphorus pentoxide.

In some embodiments, an oxygen scavenger not in contact with the matrix layer of the patch is further added to the packaging container. The oxygen scavenger comprises a main component selected from a combination of iron powder and sodium chloride, a combination of ferrous carbonate and metal halide, and a combination of ascorbate and sodium bicarbonate, preferably a combination of iron powder and sodium chloride, e.g., iron-based oxygen scavenger used in the examples.

In some embodiments, the hygroscopic agent and/or oxygen scavenger are separately packaged in a breathable film. Preferably, the hygroscopic agent and/or oxygen scavenger packaged in the breathable film are separately adhered to or placed on a heat-sealing layer in the packaging container, or are uniformly contained in the heat-sealing layer.

In some embodiments, the packaging container has low oxygen permeability and low moisture permeability, and is preferably a metal tray, a plastic tray, or a packaging bag made of a multi-layer laminated material comprising a paper layer, an aluminum foil layer and a heat-sealable inner layer.

In some embodiments, the antioxidant in the matrix is selected from ascorbic acid, ascorbyl palmitate, butylated hydroxyanisole, butylated hydroxytoluene, hypophosphorous acid, monothioglycerol, potassium metabisulfite, a small amount of propyl gallate, sodium bisulfite, sodium formaldehyde sulfoxylate, sodium metabisulfite, sodium thiosulfite, sodium thiosulfite and DL-α-tocopherol (vitamin E); the antioxidant further comprises a chelating agent as a synergistic antioxidant, including one or more of citric acid, tartaric acid, calcium disodium edetate, disodium edetate and EDTA.

In some embodiments, a combination of antioxidants in the matrix is selected from: a combination of ascorbyl palmitate, sodium metasulfite and DL-α-tocopherol (vitamin E), or a combination of ascorbyl palmitate, sodium metasulfite, DL-α-tocopherol (vitamin E) and BHT.

In some embodiments, the matrix layer comprises the following components, relative to the total weight of the matrix layer:
1) the rotigotine or pharmaceutically acceptable salt thereof at an amount of 3%-30%;
2) the stabilizer for inhibiting drug crystallization at an amount of 6%-40%;
3) the antioxidant at an amount of 0.001%-10%;
4) the pressure-sensitive adhesive at an amount of 30%-95%;
5) a tackifier at an amount of 0%-50%;
6) a skin penetration enhancer at an amount of 0%-30%;
7) a cohesion-promoting additive at an amount of 0%-20%;
the total amount of each component in the matrix layer is 100%.

In some embodiments, the pressure-sensitive adhesive is selected from one or more of an acrylic adhesive, a silicone adhesive, an acrylic-silicone copolymer adhesive, a polybutylene adhesive, a styrene-isoprene-butylene copolymer and a styrene-butadiene-styrene copolymer.

In some embodiments, the tackifier is selected from silicone oil, light mineral oil, polybutylene, triethyl citrate, terpene and a mixture thereof.

In some embodiments, the content of the rotigotine or pharmaceutically acceptable salt thereof is 5% to 20%, preferably 5% to 15%, or 5% to 12% of the total weight of the matrix layer.

In some embodiments, the content of the stabilizer for inhibiting drug crystallization is 6% to 40%, preferably 6% to 30%, 6% to 20%, 6.7%-20%, 6.70%, 8.2%, 9.2%, 10%, 12.5%, 13%, 16.65%, 17.50%, 19.00%, 20.00%, 25% and 30% of the total weight of the matrix layer.

In some embodiments, the amount of crospovidone for inhibiting drug crystallization is 6% to 40%, preferably 6% to 30%, 6% to 20%, 6.7%-20%, 6.70%, 8.2%, 9.2%, 10%, 12.5%, 13%, 16.65%, 17.50%, 19.00%, 20.00%, 25% and 30%.

In some embodiments, the amount of povidone for inhibiting drug crystallization is 0%-10%, and can be specifically selected from 0%, 1%, 2%, 3%, 4%, 5%, 6%, 7%, 8%, 9% and 10%.

In some embodiments, the content of the pressure-sensitive adhesive is 35% to 90%, preferably 40% to 90%, 70% to 90%, 65%, 70%, 75%, 80% and 90% of the total weight of the matrix layer.

In some embodiments, the amount of the tackifier is 0%-50%, preferably 0%-30% and 0%-28% of the total weight of the matrix layer.

In some embodiments, the amount of each antioxidant is 0.001%-10%, preferably 0.0015%-5%. The specific amount of each antioxidant can be selected from 0.0015%, 0.002%, 0.004%, 0.006%, 0.008%, 0.01%, 0.02%, 0.03%, 0.04%, 0.05%, 0.06%, 0.07%, 0.08%, 0.09% and 0.1%.

In some embodiments, the total amount of multiple antioxidants is 0.001%-10%, and can be specifically selected from 5%, 1%, 0.5%, 0.25%, 0.1%, 0.075%, 0.05%, 0.025%, 0.01%, 0.0075%, 0.005%, 0.0025%, 0.002% and 0.0015%.

In some embodiments, the weight ratio of rotigotine to the tackifier is 9:25-9:40, preferably 9:30-9:35.

Another aspect of the present invention provides a method for preparing the aforementioned transdermal drug delivery kit, comprising the following steps:
step 1: dissolving an antioxidant in a solvent, dissolving or suspending a stabilizer for inhibiting drug crystallization in the solvent, and mixing for 0.1 hours to 24 hours; preferably, the solvent includes but is not limited to one or more of toluene, ethanol, isopropanol, dimethylacetamide, dimethyl sulfoxide and n-heptane, and more preferably, the solvent is toluene, ethanol, isopropanol, or a mixed solvent thereof;
step 2: adding rotigotine or a pharmaceutically acceptable salt thereof, and mixing and dissolving until the rotigotine or pharmaceutically acceptable salt thereof is dispersed in a non-crystalline state;
step 3: adding a pressure-sensitive adhesive and mixing uniformly to obtain a drug wet mixture;
step 4: coating the drug wet mixture onto a release film;
step 5: drying to remove the solvent so as to obtain a release film/matrix layer laminated material;
step 6: laminating the matrix layer onto the backing layer, and performing die-cutting on a composite film to obtain individual patches;
step 7: filling a packaging container with an inert gas, and sealing and packaging the inert gas, a hygroscopic agent and/or an oxygen scavenger and the cut individual transdermal patch into the container.

In some embodiments, the solvent in step 1 includes but is not limited to one or more of toluene, ethanol, isopropanol, dimethylacetamide, dimethyl sulfoxide and n-heptane, preferably toluene, ethanol, isopropanol, n-heptane, or a mixed solvent thereof.

Unexpectedly, it has been found in the present application that during the preparation of a transdermal patch, heating a rotigotine-adhesive wet mixture to above room temperature prior to coating can obtain a patch that does not contain rotigotine crystals in a matrix layer. In contrast, if the wet mixture is not heated, crystals form in the matrix layer of the patch.

In some embodiments, the step 2 comprises the step of heating to 35 °C-65 °C for achieving a dissolved state. The heating in the step 2 is preferably performed at 45 °C-55 °C, 45 °C, 46 °C, 47 °C, 48 °C, 49 °C, 50 °C, 51 °C, 52 °C, 53 °C, 54 °C and 55 °C.

In some embodiments, the step 3 involves adding a pressure-sensitive adhesive that has been heated to 35 °C-65 °C and mixing uniformly to obtain a drug wet mixture. The heating in the step 3 is preferably performed at 45 °C-55 °C, 45 °C, 46 °C, 47 °C, 48 °C, 49 °C, 50 °C, 51 °C, 52 °C, 53 °C, 54 °C and 55 °C.

In some embodiments, the step 4 involves coating the drug wet mixture that has been heated to 35 °C-65 °C onto a release film. The heating in the step 4 is preferably performed at 45 °C-55 °C, 45 °C, 46 °C, 47 °C, 48 °C, 49 °C, 50 °C, 51 °C, 52 °C, 53 °C, 54 °C and 55 °C.

In some embodiments, the rotigotine or pharmaceutically acceptable salt thereof, the pressure-sensitive adhesive, the skin penetration enhancer and the antioxidant are pre-dried.

In some embodiments, the hygroscopic agent and/or oxygen scavenger are separately packaged in a breathable film. Preferably, the hygroscopic agent and/or oxygen scavenger packaged in the breathable film are separately adhered to or placed on a heat-sealing layer in the packaging container, or are uniformly contained in the heat-sealing layer.

Another aspect of the present invention provides the use of the aforementioned transdermal drug delivery kit in the preparation of a medicament for treating or preventing a disease sensitive to the action of a dopamine receptor agonist.

In some embodiments, the disease is a disease sensitive to the action of rotigotine.

In some embodiments, the disease is Parkinson's disease, Parkinson's plus syndrome, depression, restless legs syndrome, pain and dopaminergic neuron loss.

Another aspect of the present invention provides a method for treating or preventing a disease sensitive to the action of a dopamine receptor agonist, comprising administering a therapeutically effective amount of the aforementioned transdermal drug delivery kit to a subject in need thereof.

In a further embodiment, the rotigotine transdermal drug delivery kit is administered once every 1 day, every 3 days, or every 7 days.

In a further embodiment, the rotigotine transdermal drug delivery kit delivers about 1 mg to about 18 mg of rotigotine or a pharmaceutically acceptable salt thereof to the blood circulation system of the subject per day, preferably delivers about 1 mg to about 12 mg of rotigotine or a pharmaceutically acceptable salt thereof to the blood circulation system of the subject per day.

### DEFINITIONS

As used herein, the term “pharmaceutically acceptable salt” refers to those salts that are suitable for contact with subjects (e.g., human subjects) within reasonable medical judgment without excessive toxicity, irritation, allergic reaction, etc., have a reasonable benefit/risk ratio, and are effective for their intended use.

The “pharmaceutically acceptable salt” mentioned in the present invention includes inorganic acid addition salts and organic acid addition salts, can be prepared in situ during the final separation and purification of compounds, or can be prepared by reacting a purified compound in the form of a free base (for example, rotigotine) with an appropriate organic acid or inorganic acid separately and separating the salts thus formed.

As used herein, the term “therapeutically effective amount” refers to the amount of a compound or molecule of the present invention that, when administered to a subject, (i) treats or prevents a specific disease, disorder or condition, (ii) attenuates, improves or eliminates one or more symptoms of the specific disease, disorder or condition, or (iii) prevents or delays the onset of one or more symptoms of the specific disease, disorder or condition described herein.

As used herein, the term “about” means plus or minus 10% of the indicated number. For example, “about 10%” may represent a range of 9% to 11%, and “about 1” may represent 0.9-1.1.

As used herein, the term “treatment” refers to clinical intervention that attempts to alter the natural course of the individual being treated, and may be performed for prevention or in the course of clinical pathology. Desirable effects of treatment include, but are not limited to, preventing occurrence or recurrence of diseases, alleviating symptoms, weakening any direct or indirect pathological consequences of diseases, preventing metastasis, decreasing the rate of disease progression, ameliorating or alleviating the disease state, and reliving or improving prognosis.

As used herein, the term “insoluble crospovidone” is a high-molecular weight water-insoluble polymer obtained by subjecting N-vinyl-2-pyrrolidone to a cross-linking reaction, is a white or nearly white powder, is odorless, has good fluidity, is insoluble in water and various solvents, and is also insoluble in strong acids or strong bases. Non-limiting examples of “insoluble crospovidone” include CL-M, crospovidone CL, crospovidone CL-F and crospovidone CL-SF.

As used herein, “iron-based oxygen scavenger” is an iron-based deoxidizer, which comprises a combination of iron powder and sodium chloride as a main component.

The pressure-sensitive adhesive includes, but is not limited to, an acrylic adhesive, a silicone adhesive, an acrylic-silicone copolymer adhesive, a polybutylene adhesive, a styrene-isoprene-butylene copolymer and a styrene-butadiene-styrene copolymer, or a combination of two or more of the adhesives. Examples of acrylic adhesives are Duro-Tak® 387-2516, Duro-Tak® k 387-4280, Duro-Tak® 387-2287, Duro-Tak® 387-2510, Duro-Tak® 87-2196, Duro-Tak® 387-2051, Duro-Tak® 387-2052, Duro-Tak® 387-2054, Duro-Tak® 87-2194, Duro-Tak® 87-235A, Duro-Tak® 87-900A, Duro-Tak® 87-9301, Duro-Tak® 87-4098, GEFVA GMS® 788, GEFVA GMS® 9073, Duro-Tak® 387-2353, Duro-Tak® 87-2074, Duro-Tak® 87-2852, Duro-Tak® 87-2054, GELVA®-73 Tak® 80-1196, Duro-Tak® 87-2070, Duro-Tak® 87-2979, Duro-Tak® 87-2888 and Duro-Tak® 87-2296 from Henkel Corporation (Bridgewater, New Jersey); silicone adhesives BIO-PSA® 7-4401, BIO-PSA® 7-4402, BIO-PSA® 7-4501, BIO-PSA® 7-4502, BIO-PSA® 7-4601, BIO-PSA® 7-4602, SRS7-4502, SRS7-4501, SRS7-4502, SRS7-4602, BIO-PSA® 7-4101, BIO-PSA® 7-4102, BIO-PSA® 7-4103, BIO-PSA®7-4201, BIO-PSA® 7-4202, BIO-PSA® 7-4203, BIO-PSA® 7-4301, BIO-PSA® 7-4302, BIO-PSA® 7-4302, BIO-PSA® 7-6101, BIO-PSA® 7-6102, BIO-PSA® 7-6301 and BIO-PSA® 7-6302 from Dupont (Midland, MI). Examples of polybutylene adhesives are combinations of two or more of low-molecular weight polyisobutylenes (Oppanol B10, Oppanol B11, and Oppanol B12 from BASF), medium-molecular weight polyisobutylenes (Oppnaol B50, Oppanol B80, and Oppnaol N80), and high-molecular weight polyisobutylenes (Oppanol B100, Oppanol B150, Oppanol N100, and Oppanol N150). An example of a styrene-isoprene-butylene copolymer is D1161 (Kraton).

Crystallization inhibitors include polyvinylpyrrolidone or crosspolyvinylpyrrolidone or vinylpyrrolidone copolymers, preferably one or more of povidone K30, povidone K90, plasdone K29/32, copovidone VA64, crospovidone CL-M, hydroxypropyl methylcellulose, hydroxypropyl cellulose, hydroxypropyl methylcellulose acetate succinate, hydroxypropyl methylcellulose phthalate, hydroxypropyl betadine, a, 3, A cyclodextrin, chitosan, hyaluronic acid, pectin, carboxymethyl cellulose, alginic acid, or carrageenan.

As used herein, the term “backing layer” serves as the upper surface of a transdermal patch and provides flexibility to the patch as a main structural element. Preferably, the backing layer is substantially impermeable to a transdermally administered pharmaceutical composition. The backing layer is preferably made from a sheet or film of flexible elastic material. The backing layer is preferably air-impermeable. The backing layer used in the patch of the present invention is preferably made of a flexible, biocompatible material that mimics the elastic properties of the skin and conforms to the skin during movement. Non-occlusive backing layers allow the area to breathe (i.e. promote water vapor transmission across the skin surface), whereas occlusive backing layers reduce air/vapor penetration. Preferably, the backing layer of the matrix-type transdermal drug delivery kit is occlusive. Preferably, the backing layer contains synthetic polymers such as polyolefins, polyesters, polyethylene, polyvinylidene chloride and polyurethanes. Preferably, the thickness of the backing layer is from about 0.5 mil to about 5 mil; more preferably, the thickness of the backing layer is from about 1 mil to about 3 mils. Preferably, the oxygen transmission rate is from about 2 cc/m/24 hr to about 100 cc/m/24 hr, and more preferably, the oxygen transmission rate is from about 70 g/m/24 hr to about 90 g/m/24 hr. Preferably, the MVTR is from about 0.1 g/m/24 hr to about 50 g/m/24 hr, more preferably, the MVTR is from about 0.3 g/m/24 hr to about 30 g/m/24 hr. In a preferred embodiment, the backing layer is an occlusive polyester film layer of approximately 2.0 mil thick (commercially available as Scotchpak 9733, Scotchpak 9735 and Scotchpak 9723, 3M Drug Delivery Systems, St. Paul Minn.). Scotchpak 9733 is composed of polyester and medium density polyethylene/ethylene vinyl acetate heat-sealing layer, and the laminate is translucent, conformable, occlusive and heat-sealable. More preferably, the backing layer comprises a laminated material containing an aluminum foil layer between polymer film layers, such as Scotchpak 9738 and Scotchpak 1109. When the patch is applied to the skin, the aluminum layer prevents light from coming into contact with the photosensitive rotigotine.

As used herein, the term “release layer” includes, but is not limited to, silicone-coated polyester release liner available from many suppliers, fluoropolymer-coated polyester release liner from 3M, and fluorosilicone-coated polyester release liner.

### The Present Invention has the Advantages and Technical Effects as Follows.

1. The present inventors have obtained a transdermal patch formulation by screening, in which the crystallization of rotigotine was suppressed, thereby achieving a stable release of rotigotine.

2. The present inventors have reduced the generation of degradation impurities by adding a combination of antioxidants to a drug-matrix layer.

3. The present inventors have further reduced the generation of degradation impurities by removing air from a packaging container (e.g., a pouch) and filling with an inert gas.

4. The present inventors have reduced the impurities formed in an oxidation pathway in the rotigotine patch by packaging an antioxidant and a hygroscopic agent that are not in contact with the matrix layer of the patch, or a combination of the two. Unexpectedly, the hygroscopic agent reduces the generation of oxidative impurities. On the basis of removing air and filling with an inert gas and the hygroscopic agent, further addition of an oxygen scavenger not in contact with the patch does not result in decreased stability.

5. The inventors have found that during the preparation of a transdermal patch, heating a rotigotine-adhesive wet mixture to above room temperature prior to coating can obtain a patch that does not contain rotigotine crystals in a matrix layer.

6. The present inventors have reduced impurities by pre-drying rotigotine bulk drugs and excipients prior to mixing.

Therefore, the present invention obtains a stable rotigotine transdermal drug delivery kit, which can be stored for a long period of time under conventional conditions, has no crystallization of rotigotine dispersed in the matrix of the patch, and maintains the content of related substances at an extremely low level.

### DETAILED DESCRIPTION OF EMBODIMENTS

In order to make the objectives, technical solutions and advantages of the embodiments of the present invention clearer, the technical solutions in the embodiments of the present invention will be described clearly and completely below. Apparently, the described embodiments are module embodiments of the present invention, but not all of the embodiments. Elements and features described in one embodiment of the present invention may be combined with elements and features illustrated in one or more other embodiments. It should be noted that for purposes of clarity, representation and description of components and processes known to those of ordinary skill in the art that are not relevant to the present invention have been omitted from the description. Based on the embodiments of the present invention, all the other embodiments obtained by those of ordinary skill in the art without any inventive efforts should fall within the scope of protection of the present invention.

### ABBREVIATIONS

PVP K90=povidone K90, crospovidone=crospovidone CL-M, ascorbyl palmitate, Sodium Metasulfite is sodium metasulfite, BHT is 2,6-di-tert-butyl-p-cresol, Bio-PSA 7-4202 is a pressure-sensitive adhesive, Bio-PSA 7-4302 is a pressure-sensitive adhesive, Bio-PSA SRS7-4502 is a pressure-sensitive adhesive, Oppanol B12 SFN is a polyisobutylene adhesive, Oppanol N100 is a polyisobutylene adhesive.

### Comparative Example 1

Each component was weighed according to table 1. Soluble povidone K90 and the antioxidants α-dl-tocopherol, ascorbyl palmitate and sodium metasulfite were dissolved in ethanol, and the mixture was heated to 50 °C. Then, rotigotine was added until dissolved. The silicone adhesives Bio-PSA 7-4302 and Bio-PSA7-4202 were added and mixed uniformly. The mixed solution was coated onto a release film and dried at 40 °C for 4 minutes and at 85 °C for 4 minutes to remove the solvent. In this comparative example, the formulation patch was packaged in an aluminum foil bag containing neither nitrogen gas nor a hygroscopic agent. As shown in table 1, after storage at 40 °C for 6 months, the contents of EP impurity B, EP impurity C and EP impurity K in comparative example 1 were high.

### Comparative Example 2

Each component was weighed according to table 1. The transdermal patch of comparative example 2 was prepared using the same method as comparative example 1. In comparative example 2, the formulation patch was packaged in an aluminum foil bag containing neither nitrogen gas nor a hygroscopic agent. As shown in table 1, after storage at 40 °C for 6 months, the contents of EP impurity B, EP impurity C and EP impurity K in comparative example 2 were relatively high.

### Comparative Example 3

Each component was weighed according to the following weight ratio. 3.33% PVP K90, 0.0200% ascorbyl palmitate, 0.0009% sodium metasulfite, 0.0200% DL-α-tocopherol, 7.5% rotigotine, 13.3694% Bio-PSA 7-4302 and 75.7597% Bio-PSA 7-4202. Soluble povidone K90 and the antioxidants α-dl-tocopherol, ascorbyl palmitate and sodium metasulfite were dissolved in ethanol, and the mixture was heated to 50 °C. Then, rotigotine was added until dissolved. The silicone adhesives Bio-PSA 7-4302 and Bio-PSA7-4202 at room temperature were added and mixed uniformly. The mixed solution was coated onto a release film and dried at 40 °C for 4 minutes and at 85 °C for 4 minutes to remove the solvent. In this comparative example, the formulation patch was packaged in an aluminum foil bag containing neither nitrogen gas nor a hygroscopic agent. After storage at 40 °C for 6 months, the contents of EP impurity B, EP impurity C and EP impurity K in comparative example 3 were relatively high, which was similar to comparative examples 1-2.

In comparative examples 1-3, crystallization of rotigotine was observed after storage at room temperature, 40 °C or 60 °C for a period of time.

### Comparative Example 4

Each component was weighed according to table 1. The antioxidants α-dl-tocopherol, ascorbyl palmitate and sodium metasulfite were dissolved in ethanol, and insoluble crospovidone was added and further mixed for 24 hours. The mixed solution was heated to 50 °C, and then rotigotine was added until dissolved. The silicone adhesives Bio-PSA 7-4302 and Bio-PSA7-4202 at room temperature were added and mixed uniformly. The mixed solution was coated onto a release film and dried at 40 °C for 4 minutes and at 85 °C for 4 minutes to remove the solvent. In this comparative example, the formulation patch was packaged in an aluminum foil bag containing neither nitrogen gas nor a hygroscopic agent.

As shown in table 1, due to the high content of crospovidone contained in the formulation, the contents of EP impurity B, EP impurity C, EP impurity E and EP impurity K in comparative example 4 were higher than those in comparative examples 1, 2 and 3. The stability data of comparative examples 1-4 show that, compared with PVP K90, the use of crospovidone accelerates the degradation of rotigotine in the patch formulation, thereby reducing the stability of the patch formulation.

### Examples 1-2

Each component was weighed according to table 1. The formulation patches of examples 1-2 were prepared by a method similar to that of comparative example 3. The formulation patches of examples 1-2 were filled with nitrogen to reduce oxygen to 0.2% and packaged in aluminum foil bags. Compared with comparative examples 1-3, after being placed at 40 °C for 6 months, the contents of EP impurity B, EP impurity C and EP impurity K in the formulation patch of examples 1-2 were significantly lower. The results show that on the basis of the use of antioxidant components in a formulation patch, filling a packaging with nitrogen can further inhibit the degradation of rotigotine.

TABLE 1
Stability data of comparative examples 1-4 and examples 1-2

| | | Sample | | EP- | EP- | EP- | EP- |
|---|---|---|---|---|---|---|---|
| | Formulation | placement | | impurity | impurity | impurity | impurity |
| | information | condition | Time | B | E | C | K |
| Comparative | 4.0% PVP K90, 0.02% | Without N2 | 6 | 0.35 | 0.00 | 0.54 | 0.18 |
| example 1 | ascorbyl palmitate, | With packaging | months | | | | |
| | 0.0009% sodium | 40° C. | | | | | |
| | metasulfite, 0.02% DL- | | | | | | |
| | α-tocopherol, 9.000% | | | | | | |
| | rotigotine, 52.1755% | | | | | | |
| | Bio-PSA 7-4302 and | | | | | | |
| | 34.7836% Bio-PSA 7- | | | | | | |
| | 4202 | | | | | | |
| Comparative | 5.0% PVP K90, 0.02% | Without N2 | 6 | 0.46 | 0.00 | 0.67 | 0.15 |
| example 2 | ascorbyl palmitate, | With packaging | months | | | | |
| | 0.0009% sodium | 40° C. | | | | | |
| | metasulfite, 0.02% DL- | | | | | | |
| | α-tocopherol, 9.000% | | | | | | |
| | rotigotine, 51.5755% | | | | | | |
| | Bio-PSA 7-4302 and | | | | | | |
| | 34.3836% Bio-PSA 7- | | | | | | |
| | 4202 | | | | | | |
| Comparative | 16.65% crospovidone, | Without N2 | 6 | 1.26 | 0.09 | 1.53 | 0.68 |
| example 4 | 0.0200% ascorbyl | With packaging | months | | | | |
| | palmitate, 0.0009% | 40° C. | | | | | |
| | sodium metasulfite, | | | | | | |
| | 0.0200% vitamin E, | | | | | | |
| | 7.5% rotigotine and | | | | | | |
| | 75.8091% Bio-PSA 7- | | | | | | |
| | 4302 | | | | | | |
| Example 1 | 3.3332% PVP K90, | With N2 | 5 | 0.00 | 0.00 | 0.04 | 0.01 |
| | 0.0167% ascorbyl | With packaging | days | | | | |
| | palmitate, 0.0015% | 60° C. | 10 | 0.00 | 0.00 | 0.05 | 0.01 |
| | sodium metasulfite, | | days | | | | |
| | 0.0417% DL-α- | With N2 | 3 | 0.00 | 0.00 | 0.04 | 0.02 |
| | tocopherol, 7.5000% | With packaging | months | | | | |
| | rotigotine, 53.4641% | 40° C. | 6 | 0.04 | 0.02 | 0.06 | 0.02 |
| | Bio-PSA 7-4302 and | | months | | | | |
| | 35.6428% Bio-PSA 7- | With N2 | 3 | 0.00 | 0.00 | 0.02 | 0.01 |
| | 4202 | With packaging | months | | | | |
| | | RT | 6 | 0.02 | 0.04 | 0.05 | 0.01 |
| | | | months | | | | |
| Example 2 | 4.0000% PVP K90, | With N2 | 5 | 0.00 | 0.00 | 0.04 | 0.01 |
| | 0.0167% ascorbyl | With packaging | days | | | | |
| | palmitate, 0.0015% | 60° C. | 10 | 0.04 | 0.00 | 0.05 | 0.01 |
| | sodium metasulfite, | | days | | | | |
| | 0.0417% DL-α- | With N2 | 3 | 0.00 | 0.00 | 0.04 | 0.01 |
| | tocopherol, 9.0000% | With packaging | months | | | | |
| | rotigotine, 47.8171% | 40° C. | 6 | 0.02 | 0.02 | 0.07 | 0.02 |
| | Bio-PSA 7-4302 and | | months | | | | |
| | 39.1230% Bio-PSA 7- | With N2 | 3 | 0.00 | 0.00 | 0.04 | 0.01 |
| | 4202 | With packaging | months | | | | |
| | | RT | 6 | 0.04 | 0.05 | 0.05 | 0.01 |
| | | | months | | | | |

### Comparative Example 5 and Examples 3-6

Each component was weighed according to table 1. Soluble povidone K90 and the antioxidants α-dl-tocopherol, ascorbyl palmitate and sodium metasulfite were dissolved in ethanol, and crospovidone was added and further mixed for 24 hours. The mixed solution was heated to 50 °C, and then rotigotine was added until dissolved. The silicone adhesives Bio-PSA 7-4302 and Bio-PSA7-4202 at room temperature were added and mixed uniformly. The suspension was coated onto a release film and dried at 40 °C for 4 minutes and at 85 °C for 4 minutes to remove the solvent. The formulation patches were packaged using different packaging methods and placed at 60 °C for 5 days. The related substances of comparative example 5 and examples 3-6 were examined. The experimental conclusions are summarized in table 2.

The formulation patch of comparative example 5 was packaged using only an aluminum foil bag.

The formulation patch of example 3 was filled with nitrogen to reduce oxygen to 0.2% and packaged in an aluminum foil bag. Compared with comparative example 5, the contents of EP impurity B, EP impurity C and EP impurity K in example 3 were reduced.

With regard to the formulation patch of example 4, an iron-based oxygen scavenger not in contact with the patch was placed in a packaging bag, nitrogen was filled to reduce oxygen to 0.2%, and the patch and iron-based oxygen scavenger were packaged in an aluminum foil bag. There is no difference in impurity contents between example 4 and example 3. On the basis of filling a packaging with nitrogen, further addition of an iron-based oxygen scavenger does not result in decreased stability.

With regard to the formulation patch of example 5, a hygroscopic film bag containing hygroscopic calcium oxide was placed in a packaging bag, nitrogen was filled to reduce oxygen to 0.2%, and the patch and hygroscopic film bag were packaged in an aluminum foil bag. On the basis of filling a packaging with nitrogen, further addition of a hygroscopic film bag can inhibit the generation of some impurities.

With regard to the formulation patch of example 6, a hygroscopic film bag containing hygroscopic calcium oxide and an iron-based oxygen scavenger were placed in a packaging bag, nitrogen was filled to reduce oxygen to 0.2%, and the patch, hygroscopic film bag and iron-based oxygen scavenger were packaged in an aluminum foil bag. On the basis of filling a packaging with nitrogen, further addition of a hygroscopic film bag and an iron-based oxygen scavenger does not result in decreased stability.

The data in table 2 show that there is little difference in degradation impurity contents among examples 3, 4, 5 and 6. In the transdermal patch formulation using both PVP K90 and cross-linked PVP as crystallization inhibitors, if the amount of the cross-linked PVP is not large, the use of an aluminum foil packaging filled with N2 can inhibit the degradation of rotigotine. On the basis of filling with N2, further addition of a hygroscopic agent, an iron-based oxygen scavenger, or a combination of a hygroscopic agent and an iron-based oxygen scavenger does not result in decreased stability.

TABLE 2
Stability data of comparative example 5 and examples 3-6

| | | | | EP- | EP- | EP- | EP- |
|---|---|---|---|---|---|---|---|
| | Formulation | Sample placement | | impurity | impurity | impurity | impurity |
| | information | condition | Time | B | E | C | K |
| Comparative | 2.500% PVP K90, | Without N2 | 5 | 0.09 | 0.00 | 0.22 | 0.15 |
| example | 6.700% | With packaging | days | | | | |
| 5 | crospovidone, | 60° C. | | | | | |
| Example | 0.0167% ascorbyl | With N2 | 5 | 0.07 | 0.05 | 0.08 | 0.10 |
| 3 | palmitate, 0.0015% | With packaging | days | | | | |
| | sodium metasulfite, | 60° C. | | | | | |
| Example | 0.0583% DL-α- | With N2 | 5 | 0.06 | 0.05 | 0.06 | 0.09 |
| 4 | tocopherol, | With packaging | days | | | | |
| | 7.5000% rotigotine, | Iron-based oxygen | | | | | |
| | 49.9341% Bio-PSA | scavenger | | | | | |
| | 7-4302 and | 60° C. | | | | | |
| Example | 33.2894% Bio-PSA | With N2 | 5 | 0.06 | 0.13 | 0.05 | 0.08 |
| 5 | 7-4202 | With packaging | days | | | | |
| | | CaO | | | | | |
| | | 60° C. | | | | | |
| Example | | With N2 | 5 | 0.06 | 0.09 | 0.08 | 0.10 |
| 6 | | With packaging | days | | | | |
| | | Iron-based oxygen | | | | | |
| | | scavenger | | | | | |
| | | CaO | | | | | |
| | | 60° C. | | | | | |

### Comparative Examples 6-10 and Examples 7-9

Each component of a composition was weighed according to table 3, and the formulation was prepared. The antioxidants ascorbyl palmitate, sodium metasulfite and α-dl-tocopherol were dissolved in isopropanol at 50 °C. Insoluble crospovidone was added and mixed at room temperature for 24 hours. The suspension was heated to 50 °C, and rotigotine was added until completely dissolved. A solution of light mineral oil and polyisobutylene adhesives Oppanol B12 and Oppanol N100 in n-heptane heated to 50 °C was added and mixed. The suspension was kept at 50 °C, coated onto a release film, and dried at 40 °C for 4 min and at 85 °C for 4 min to remove the solvent.

In comparative examples 6-10 and examples 7-9, only crospovidone was used as a crystallization inhibitor for rotigotine.

In the aluminum foil bags of comparative examples 6-10, neither nitrogen nor a hygroscopic agent was used. As shown in table 3, after storage at 40 °C for 3 months, the contents of EP impurity B, EP impurity C, EP impurity E and EP impurity K in comparative examples 6-10 increased significantly.

The patch formulations of examples 7-9 were filled with nitrogen to reduce oxygen to 0.2% and packaged in aluminum foil bags. As shown in table 3, after storage at 60 °C, 40 °C and room temperature for a period of time, the contents of EP impurity B, EP impurity C, EP impurity E and EP impurity K in examples 7-9 were lower than those in comparative examples 6-10. The results show that filling a packaging with nitrogen can inhibit the degradation of rotigotine in a formulation in which only crospovidone was used.

TABLE 3
Stability data of comparative examples 6-9 and examples 7-9

| | | Sample | | EP- | EP- | EP- | EP- |
|---|---|---|---|---|---|---|---|
| | Formulation | placement | | impurity | impurity | impurity | impurity |
| | information | condition | Time | B | E | C | K |
| Comparative | 20.0000% | With | 3 | 0.89 | 0.05 | 1.24 | 0.55 |
| example 6 | crospovidone, | packaging | months | | | | |
| | 0.0200% ascorbyl | 40° C. | | | | | |
| | palmitate, 0.0009% | Without N2 | | | | | |
| | sodium metasulfite, | | | | | | |
| | 0.0200% DL-α- | | | | | | |
| | tocopherol, | | | | | | |
| | 28.3836% light | | | | | | |
| | mineral oil, 25.5453% | | | | | | |
| | Oppanol B12, | | | | | | |
| | 17.0302% Oppanol | | | | | | |
| | N100 and 9.0000% | | | | | | |
| | rotigotine | | | | | | |
| Comparative | 27.0000% | With | 3 | 0.70 | 0.00 | 1.18 | 1.94 |
| example 7 | crospovidone, | packaging | months | | | | |
| | 0.0200% ascorbyl | 40° C. | | | | | |
| | palmitate, 0.0009% | Without N2 | | | | | |
| | sodium metasulfite, | | | | | | |
| | 0.0200% DL-α- | | | | | | |
| | tocopherol, | | | | | | |
| | 25.5836% light | | | | | | |
| | mineral oil, 23.0253% | | | | | | |
| | Oppanol B12, | | | | | | |
| | 15.3502% Oppanol | | | | | | |
| | N100 and 9.0000% | | | | | | |
| | rotigotine | | | | | | |
| Comparative | 16.6500% | With | 3 | 0.70 | 0.07 | 0.70 | 0.57 |
| example 8 | crospovidone, | packaging | months | | | | |
| | 0.0167% ascorbyl | Without N2 | | | | | |
| | palmitate, 0.0015% | 40° C. | | | | | |
| | sodium metasulfite, | | | | | | |
| | 0.0417% DL-α- | | | | | | |
| | tocopherol, | | | | | | |
| | 26.5265% light | | | | | | |
| | mineral oil, 29.5581% | | | | | | |
| | Oppanol B12, | | | | | | |
| | 19.7054% Oppanol | | | | | | |
| | N100 and 7.5000% | | | | | | |
| | rotigotine | | | | | | |
| Comparative | 10.0000% | With | 6 | 0.74 | 0.04 | 0.89 | 0.69 |
| example 9 | crospovidone, | packaging | months | | | | |
| | 0.0167% ascorbyl | Without N2 | | | | | |
| | palmitate, 0.0015% | 40° C. | | | | | |
| | sodium metasulfite, | | | | | | |
| | 0.0417% DL-α- | | | | | | |
| | tocopherol, 7.5000% | | | | | | |
| | rotigotine, | | | | | | |
| | 28.8540% light | | | | | | |
| | mineral oil, 32.1516% | | | | | | |
| | Oppanol B12, | | | | | | |
| | 21.4344% Oppanol | | | | | | |
| | N100 | | | | | | |
| Example 7 | 16.6500% | With N2 | 5 | 0.07 | 0.65 | 0.23 | 0.23 |
| | crospovidone, | With | days | | | | |
| | 0.0167% ascorbyl | packaging | | | | | |
| | palmitate, 0.0015% | 60° C. | | | | | |
| | sodium metasulfite, | With N2 | 1 | 0.04 | 0.29 | 0.55 | 0.20 |
| | 0.0583% DL-α- | With | months | | | | |
| | tocopherol, 7.5000% | packaging | | | | | |
| | rotigotine, | 40° C. | | | | | |
| | 26.5207% light | | | | | | |
| | mineral oil, 29.5517% | With N2 | 6 | 0.17 | 0.15 | 0.30 | 0.28 |
| | Oppanol B12 and | With | months | | | | |
| | 19.7011% Oppanol | packaging | | | | | |
| | N100 | RT | | | | | |
| Example 8 | 20.00% | With N2 | 5 | 0.07 | 0.55 | 0.27 | 0.26 |
| | crospovidone, | With | days | | | | |
| | 0.0167% ascorbyl | packaging | | | | | |
| | palmitate, | 60° C. | | | | | |
| | 0.0015% sodium | With N2 | 1 | 0.17 | 0.09 | 0.80 | 0.27 |
| | metasulfite, 0.0583% | With | months | | | | |
| | DL-α-tocopherol, | packaging | | | | | |
| | 7.5000% rotigotine, | 40° C. | | | | | |
| | 25.3482% light | | | | | | |
| | mineral oil, 28.2452% | With N2 | 6 | 0.50 | 0.11 | 0.77 | 0.45 |
| | Oppanol B12 and | With | months | | | | |
| | 18.8301% Oppanol | packaging | | | | | |
| | N100 | RT | | | | | |
| Example 9 | 13% crospovidone, | With N2 | 5 | 0.02 | 0.22 | 0.17 | 0.22 |
| | 0.0167% ascorbyl | With | days | | | | |
| | palmitate, | packaging | | | | | |
| | 0.0015% sodium | 60° C. | | | | | |
| | metasulfite, 0.0583% | With N2 | 1 | 0.02 | 0.11 | 0.17 | 0.20 |
| | DL-α-tocopherol, | With | months | | | | |
| | 7.5000% rotigotine, | packaging | | | | | |
| | 27.7982% light | 40° C. | | | | | |
| | mineral oil, 30.9752% | With N2 | 6 | 0.19 | 0.04 | 0.39 | 0.30 |
| | Oppanol B12 and | With | months | | | | |
| | 20.6501% Oppanol | packaging | | | | | |
| | N100 | RT | | | | | |

### Examples 10-12

Each component of a composition was weighed according to table 4, and the formulation was prepared. The antioxidants ascorbyl palmitate, sodium metasulfite, DL-α-tocopherol and BHT were dissolved in isopropanol at 50 °C. Insoluble crospovidone was added and mixed at room temperature for 24 hours. The suspension was heated to 50 °C, and rotigotine was added until completely dissolved. A solution of light mineral oil and polyisobutylene adhesives Oppanol B12 and Oppanol N100 in n-heptane heated to 50 °C was added and mixed. The suspension was kept at 50 °C, coated onto a release film, and dried at 40 °C for 4 min and at 85 °C for 4 min to remove the solvent.

The formulation patch of example 10 was filled with nitrogen to reduce oxygen to 0.2% and packaged in an aluminum foil bag. After storage at 60 °C, 40 °C and room temperature for a period of time, the contents of EP impurity B, EP impurity C and EP impurity K were maintained at a relatively low level.

In example 11, a hygroscopic film bag containing silica gel was placed in a packaging bag, nitrogen was filled to reduce oxygen to 0.2%, and the patch and the silica gel-containing hygroscopic film bag were packaged in an aluminum foil bag. On the basis of filling a packaging with nitrogen, further addition of a silica gel-containing hygroscopic film bag can further inhibit the generation of impurities.

In example 12, a hygroscopic film bag containing hygroscopic calcium chloride was placed in a packaging bag, nitrogen was filled to reduce oxygen to 0.2%, and the patch and hygroscopic film bag were packaged in an aluminum foil bag. On the basis of filling a packaging with nitrogen, the addition of a hygroscopic film bag containing hygroscopic calcium chloride can further inhibit the generation of impurities.

Compared with example 10, a hygroscopic agent was used in examples 11-12, which further inhibited the generation of degradation impurities, especially oxidative degradation impurities and the secondary degradation impurities thereof.

TABLE 4
Stability data of examples 10-12

| | | | | EP- | EP- | EP- | EP- |
|---|---|---|---|---|---|---|---|
| | | Sample placement | | impurity | impurity | impurity | impurity |
| | | condition | Time | B | E | C | K |
| Example | 16.6500% | With N2 | 5 days | 0.00 | 0.33 | 0.13 | 0.25 |
| 10 | crospovidone, | With packaging | | | | | |
| | 0.0167% ascorbyl | 60° C. | | | | | |
| | palmitate, | With N2 | 1 | 0.09 | 0.33 | 0.08 | 0.25 |
| | 0.0015% sodium | With packaging | month | | | | |
| | metasulfite, | 40°C | | | | | |
| | 0.0583% DL-α- | With N2 | 3 | 0.07 | 0.24 | 0.07 | 0.20 |
| | tocopherol, | With packaging | months | | | | |
| | 0.0500% BHT, | RT | | | | | |
| Example | 7.5000% | With N2 | 5 days | 0.00 | 0.25 | 0.08 | 0.17 |
| 11 | rotigotine, | With packaging | | | | | |
| | 26.5032% light | 2 g of silica gel | | | | | |
| | mineral oil, | 60° C. | | | | | |
| | 29.5322% | With N2 | 1 | 0.09 | 0.24 | 0.10 | 0.15 |
| | Oppanol B12, | With packaging | month | | | | |
| | 19.881% Oppanol | 2 g of silica gel | | | | | |
| | N100 | 40° C. | | | | | |
| | | With N2 | 3 | 0.07 | 0.16 | 0.08 | 0.11 |
| | | With packaging | months | | | | |
| | | 2 g of silica gel | | | | | |
| | | RT | | | | | |
| Example | | With N2 | 5 days | 0.00 | 0.22 | 0.08 | 0.15 |
| 12 | | With packaging | | | | | |
| | | 2 g of CaCl2 | | | | | |
| | | 60° C. | | | | | |
| | | With N2 | 1 | 0.07 | 0.24 | 0.08 | 0.14 |
| | | With packaging | month | | | | |
| | | 2 g of CaCl2 | | | | | |
| | | 40° C. | | | | | |
| | | With N2 | 3 | 0.07 | 0.16 | 0.07 | 0.12 |
| | | With packaging | months | | | | |
| | | 2 g of CaCl2 | | | | | |
| | | RT | | | | | |

### Comparative Example 11 and Examples 13-16

Each component was weighed according to table 5. The antioxidants ascorbyl palmitate, sodium metasulfite and DL-α-tocopherol were dissolved in isopropanol at 50 °C. Insoluble crospovidone was added and mixed at room temperature for 24 hours. The suspension was heated to 50 °C, and rotigotine was added until completely dissolved. A solution of light mineral oil and polyisobutylene adhesives Oppanol B12 and Oppanol N100 in n-heptane heated to 50 °C was added and mixed. The suspension was kept at 50 °C, coated onto a release film, and dried at 40 °C for 4 min and at 85 °C for 4 min to remove the solvent.

The formulation patches were packaged using different packaging methods and placed at 60 °C for 5 days. The related substances of comparative example 11 and examples 13-16 were examined. The experimental conclusions are summarized in table 5.

The formulation patch of comparative example 11 was packaged using only an aluminum foil bag.

The formulation patch of example 13 was filled with nitrogen to reduce oxygen to 0.2% and packaged in an aluminum foil bag. Compared with comparative example 11, the contents of EP impurity B, EP impurity C and EP impurity K in example 13 were reduced.

With regard to the formulation patch of example 14, an iron-based oxygen scavenger was placed in a packaging bag, nitrogen was filled to reduce oxygen to 0.2%, and the patch and iron-based oxygen scavenger were packaged in an aluminum foil bag. There is no difference in impurity contents between example 14 and example 13. On the basis of filling a packaging with nitrogen, further addition of an iron-based oxygen scavenger does not result in decreased stability.

With regard to the formulation patch of example 15, a hygroscopic film bag containing hygroscopic calcium oxide was placed in a packaging bag, nitrogen was filled to reduce oxygen to 0.2%, and the patch and hygroscopic film bag were packaged in an aluminum foil bag. On the basis of filling a packaging with nitrogen, further addition of a hygroscopic film bag can inhibit the generation of four impurities.

With regard to the formulation patch of example 16, a hygroscopic film bag containing hygroscopic calcium oxide and an iron-based oxygen scavenger were placed in a packaging bag, nitrogen was filled to reduce oxygen to 0.2%, and the patch, hygroscopic film bag and iron-based oxygen scavenger were packaged in an aluminum foil bag. The levels of related impurities of example 16 and example 15 were similar. It shows that on the basis of filling a packaging with nitrogen, further addition of a hygroscopic film bag and an iron-based oxygen scavenger does not result in decreased stability.

The data in table 5 show that in a transdermal patch formulation using only cross-linked PVP as a rotigotine crystallization inhibitor, the use of an aluminum foil packaging filled with N2 can inhibit the degradation of rotigotine. If the amount of cross-linked PVP is large, on the basis of filling with N2, the addition of a hygroscopic agent can further inhibit the degradation of rotigotine. Further addition of an iron-based oxygen scavenger to a packaging does not result in decreased stability.

TABLE 5
Stability data of comparative example 11 and examples 13-16

| | | Sample | | EP- | EP- | EP- | EP- |
|---|---|---|---|---|---|---|---|
| | Formulation | placement | | impurity | impurity | impurity | impurity |
| | information | condition | Time | B | E | C | K |
| Comparative | 17.5000% | Without N2 | 5 | 0.41 | 0.05 | 0.35 | 0.29 |
| example 11 | crospovidone, | With packaging | days | | | | |
| | 0.0167% ascorbyl | 60° C. | | | | | |
| | palmitate, | | | | | | |
| Example 13 | 0.0015% sodium | N2 | 5 | 0.17 | 0.18 | 0.13 | 0.23 |
| | metasulfite, | With packaging | days | | | | |
| | 0.0583% DL-α- | 60° C. | | | | | |
| Example 14 | tocopherol, | With N2 | 5 | 0.19 | 0.18 | 0.13 | 0.23 |
| | 7.5000% | With packaging | days | | | | |
| | rotigotine, | Iron-based | | | | | |
| | 26.2232% light | oxygen | | | | | |
| | mineral oil, | scavenger | | | | | |
| | 29.2202% | 60° C. | | | | | |
| Example 15 | Oppanol B12 and | With N2 | 5 | 0.13 | 0.15 | 0.10 | 0.14 |
| | 19.4801% | With packaging | days | | | | |
| | Oppanol N100 | CaO | | | | | |
| | | 60° C. | | | | | |
| Example 16 | | With N2 | 5 | 0.13 | 0.15 | 0.11 | 0.14 |
| | | With packaging | days | | | | |
| | | Iron-based | | | | | |
| | | oxygen | | | | | |
| | | scavenger | | | | | |
| | | CaO | | | | | |
| | | 60° C. | | | | | |

1. A rotigotine transdermal drug delivery kit, comprising a transdermal drug delivery patch, wherein the transdermal drug delivery patch comprises:
1) a backing layer;
2) a matrix layer, comprising rotigotine or a pharmaceutically acceptable salt thereof that is dispersed in the matrix layer in a non-crystalline state, a stabilizer for inhibiting drug crystallization, an antioxidant and a pressure-sensitive adhesive; and
3) a release layer;
wherein the transdermal drug delivery patch is packaged in a packaging container.

2. The transdermal drug delivery kit according to claim 1, wherein the packaging container is filled with an inert gas; the inert gas is preferably nitrogen; preferably, the inert gas is filled to reduce oxygen to below about 3.0%; more preferably, nitrogen is filled to reduce oxygen to below about 2.0%, 1.0%, 0.5%, 0.2%, 0.15%, 0.1%, 0.05%, or 0.01%.

3. The transdermal drug delivery kit according to claim 1 or 2, wherein the stabilizer for inhibiting drug crystallization contains a peroxide; preferably, the peroxide-containing stabilizer for inhibiting drug crystallization comprises insoluble crospovidone; further preferably, the insoluble crospovidone is selected from one or more of insoluble crospovidone CL-M, crospovidone CL, crospovidone CL-F and crospovidone CL-SF.

4. The transdermal drug delivery kit according to any one of claims 1-3, wherein in addition to the insoluble crospovidone, the stabilizer for inhibiting drug crystallization further comprises soluble povidone, wherein the soluble povidone is preferably one or more of povidone K30, povidone K90, povidone K12, povidone K17, povidone K25, plasdone K29/32, and copovidone VA64.

5. The transdermal drug delivery kit according to any one of claims 1-4, wherein a hygroscopic agent not in contact with the matrix layer of the patch is further added to the packaging container.

6. The transdermal drug delivery kit according to claim 5, wherein the hygroscopic agent is selected from one or more of calcium chloride, calcium oxide, barium oxide, calcium sulfate, molecular sieve, sodium sulfate, magnesium sulfate, silicon dioxide, silica gel, potassium hydroxide, potassium carbonate and phosphorus pentoxide.

7. The transdermal drug delivery kit according to any one of claims 1-6, wherein an oxygen scavenger not in contact with the matrix layer of the patch is further added to the packaging container.

8. The transdermal drug delivery kit according to claim 7, wherein the oxygen scavenger not in contact with the matrix layer of the patch comprises a main component selected from a combination of iron powder and sodium chloride, a combination of ferrous carbonate and metal halide, and a combination of ascorbate and sodium bicarbonate, preferably a combination of iron powder and sodium chloride.

9. The transdermal drug delivery kit according to any one of claims 1-8, wherein the hygroscopic agent and/or oxygen scavenger are separately packaged in a breathable film; preferably, the hygroscopic agent and/or oxygen scavenger packaged in the breathable film are separately adhered to or placed on a heat-sealing layer in the packaging container, or are uniformly contained in the heat-sealing layer.

10. The transdermal drug delivery kit according to any one of claims 1-9, wherein the packaging container has low oxygen permeability and low moisture permeability, and is preferably a metal tray, a plastic tray, or a packaging bag made of a multi-layer laminated material comprising a paper layer, an aluminum foil layer and a heat-sealable inner layer.

11. The transdermal drug delivery kit according to any one of claims 1-10, wherein the antioxidant is selected from ascorbic acid, ascorbyl palmitate, butylated hydroxyanisole, butylated hydroxytoluene, hypophosphorous acid, monothioglycerol, potassium metabisulfite, propyl gallate, sodium bisulfite, sodium formaldehyde sulfoxylate, sodium metabisulfite, sodium thiosulfite, sodium thiosulfite and DL-α-tocopherol; preferably, the antioxidant further comprises a chelating agent as a synergistic antioxidant, including one or more of citric acid, tartaric acid, calcium disodium edetate, disodium edetate and EDTA; the antioxidant is preferably a combination of ascorbyl palmitate, sodium metasulfite and DL-α-tocopherol, or a combination of ascorbyl palmitate, sodium metasulfite, DL-α-tocopherol and BHT.

12. The transdermal drug delivery kit according to any one of claims 1-11, wherein the matrix layer comprises the following components, relative to the total weight of the matrix layer:
1) the rotigotine or pharmaceutically acceptable salt thereof at an amount of 3%-30%;
2) the stabilizer for inhibiting drug crystallization at an amount of 6%-40%;
3) the antioxidant at an amount of 0.001%-10%;
4) the pressure-sensitive adhesive at an amount of 30%-95%;
5) a tackifier at an amount of 0%-50%;
6) a skin penetration enhancer at an amount of 0%-30%;
7) a cohesion-promoting additive at an amount of 0%-20%;
the total amount of each component in the matrix layer is 100%.

13. The transdermal drug delivery kit according to any one of claims 1-12, wherein the pressure-sensitive adhesive is selected from one or more of an acrylic adhesive, a silicone adhesive, an acrylic-silicone copolymer adhesive, a polybutylene adhesive, a styrene-isoprene-butylene copolymer and a styrene-butadiene-styrene copolymer.

14. The transdermal drug delivery kit according to any one of claims 12 and 13, wherein the tackifier is selected from silicone oil, light mineral oil, polybutylene, triethyl citrate, terpene and a mixture thereof.

15. A method for preparing the transdermal drug delivery kit according to any one of claims 1-14, comprising the following steps:
step 1: dissolving an antioxidant in a solvent, dissolving or suspending a stabilizer for inhibiting drug crystallization in the solvent, and mixing for 0.1 hours to 24 hours; preferably, the solvent includes but is not limited to one or more of toluene, ethanol, isopropanol, dimethylacetamide, dimethyl sulfoxide and n-heptane, and more preferably, the solvent is toluene, ethanol, isopropanol, n-heptane, or a mixed solvent thereof;
step 2: adding rotigotine or a pharmaceutically acceptable salt thereof, and mixing and dissolving until the rotigotine or pharmaceutically acceptable salt thereof is dispersed in a non-crystalline state;
step 3: adding a pressure-sensitive adhesive and mixing uniformly to obtain a drug wet mixture;
step 4: coating the drug wet mixture onto a release film;
step 5: drying to remove the solvent so as to obtain a release film/matrix layer laminated material;
step 6: laminating the matrix layer onto the backing layer, and performing die-cutting on a composite film to obtain individual patches;
step 7: filling a packaging container with an inert gas, and sealing and packaging the inert gas, a hygroscopic agent and/or an oxygen scavenger and the cut individual transdermal patch into the container.

16. The method according to claim 15, wherein the step 2 comprises the step of heating to 35 °C-65 °C for achieving a dissolved state of a drug; the heating is preferably performed at 45 °C-55 °C.

17. The method according to claim 15 or 16, wherein the step 3 involves adding a pressure-sensitive adhesive that has been heated to 35 °C-65 °C and mixing uniformly to obtain a drug wet mixture; the heating is preferably performed at 45 °C-55 °C.

18. The method according to any one of claims 15-17, wherein the step 4 involves coating the drug wet mixture that has been heated to 35 °C-65 °C onto a release film; the heating is preferably performed at 45 °C-55 °C.

19. The method according to any one of claims 15-18, wherein the rotigotine or pharmaceutically acceptable salt thereof, the pressure-sensitive adhesive, the skin penetration enhancer and the antioxidant are pre-dried.

20. The method according to any one of claims 15-19, wherein the hygroscopic agent and/or oxygen scavenger are separately packaged in a breathable film; preferably, the hygroscopic agent and/or oxygen scavenger packaged in the breathable film are separately adhered to or placed on a heat-sealing layer in the packaging container, or are uniformly contained in the heat-sealing layer.

21. Use of the transdermal drug delivery kit according to any one of claims 1-20 in the preparation of a medicament for treating or preventing a disease sensitive to the action of a dopamine receptor agonist.

22. The use according to claim 21, wherein the disease is Parkinson's disease, Parkinson's plus syndrome, depression, restless legs syndrome, pain and dopaminergic neuron loss.

23. A method for treating or preventing a disease sensitive to the action of a dopamine receptor agonist, comprising administering a therapeutically effective amount of the transdermal drug delivery kit according to any one of claims 1-20 to a subject in need thereof.

24. The use according to claim 23, wherein the disease is Parkinson's disease, Parkinson's plus syndrome, depression, restless legs syndrome, pain and dopaminergic neuron loss.
